(19) United States
(12) Patent Application Publication
(10) Pub. No.: US 2014/0249102 A1
(43) Pub. Date: Sep. 4, 2014

(54) Topical Applicator Composition and Process for Treatment of Radiologically Contaminated Dermal Injuries

(71) Applicants: Tatiana G. Levitskaia, Kennewick, WA (US); Karla D. Thrall, West Richland, WA (US); James M. Peterson, West Richland, WA (US); Glen E. Fryxell, Kennewick, WA (US); Charles A. Timchalk, Kennewick, WA (US); Barbara J. Tarasevich, Richland, WA (US)

(72) Inventors: Tatiana G. Levitskaia, Kennewick, WA (US); Karla D. Thrall, West Richland, WA (US); James M. Peterson, West Richland, WA (US); Glen E. Fryxell, Kennewick, WA (US); Charles A. Timchalk, Kennewick, WA (US); Barbara J. Tarasevich, Richland, WA (US)

(73) Assignee: BATTELLE MEMORIAL INSTITUTE, Richland, WA (US)

(21) Appl. No.: 13/173,262

(22) Filed: Jun. 30, 2011

Related U.S. Application Data
(60) Provisional application No. 61/379,175, filed on Sep. 1, 2010.

(51) Int. Cl. A61K 31/722; A61K 9/00; A61K 45/06; A61K 31/734

(52) U.S. Cl. CPC A61K 31/722; A61K 31/734; A61K 9/0014; A61K 45/06
USPC 514/55; 514/54

(57) ABSTRACT

A topical applicator composition and process are described that decorporate radionuclides from radiologically-contaminated dermal surfaces and that further promote healing. The topical applicator includes a decorporation agent mixed with a plasticizing agent that forms a covering when applied to the dermal surface that decorporates radionuclides and minimizes their systemic migration. The topical applicator formulations can be delivered in conjunction with bandages and other application dressings.

### STATEMENT REGARDING RIGHTS TO INVENTION MADE UNDER FEDERALLY-SPONSORED RESEARCH AND DEVELOPMENT

This invention was made with Government support under Contract DE-AC05-76RLO1830 awarded by the U.S. Department of Energy. The Government has certain rights in the invention.

### FIELD OF THE INVENTION

The present invention relates generally to topical skin dressings for treatment of dermal wounds and burns. More particularly, the invention is a topical applicator composition and process that includes mixed polysaccharides that decorporate radionuclides from radiologically-contaminated dermal injuries and promotes healing.

### BACKGROUND OF THE INVENTION

Current events have highlighted various terrorist actions that may eventually be intended to set up a nuclear explosion or disseminate radioactive materials using a radiological dispersal device. In such an event, population exposure to large doses of external and/or internal ionizing radiation is likely, which will include traumatic injuries. For example, radioactive fallout resulting from a nuclear emergency or other radiological event can be expected to result in radiological contamination and localized cutaneous radiation injury. Cutaneous Radiation Combined Injury (CRCI) is defined as a radiologically contaminated thermal burn, or a mechanical or chemical burn resulting, e.g., from weapons of mass destruction (WMD), nuclear explosions, or other radiological contamination incidents. In the event of a nuclear explosion, cutaneous radiation injuries are predicted to account for the majority of all injuries, in which multiple radioisotopes can be deposited onto the injured skin. If left untreated, radiologically contaminated burns and wounds from injuries serve as an entry point for radionuclides that can cause internal systemic contamination. In a non-nuclear war zone, contamination of cutaneous injuries with depleted uranium (DU) is a major health concern. To date, mechanisms and health effects of systemic radionuclide uptake from dermal wounds have been investigated to only a limited extent, and no specific treatment options are currently available. Thus, the exploration of consequences and treatment of radiologically contaminated cutaneous injuries has been recently identified as a high priority research area in order to develop counter-measures for radiological and nuclear emergencies.

Wound healing is a complicated process involving cell proliferation, migration, and tissue reconstruction. Three distinct injury zones are present in a burn injury site: 1) a coagulation zone, where tissue is permanently destroyed and blood flow ceases; 2) a stasis zone, where blood flow in a tissue ceases during the first day post-burn; and 3) a hyperemia zone, where an extensive burn causes a systemic response due to loss of the skin barrier, including, e.g., release of vasoactive mediators from the wound, and a subsequent infection. Interstitial edema can also lead to multiple organ failures with, e.g., an initial decrease in cardiac output and a decrease in the Metabolic rate of multiple organs and soft tissues remote from the original burn wound. After successful recovery from this stage, a hypermetabolic phase takes place. Burn patients experience a prolonged period of inflammatory response with a concomitant elevated generation of free radicals, which free radicals can cause systemic tissue damage. In addition thermal injury can initiate a systemic inflammatory response that produces inflammatory reactions, burn toxins, reactive oxygen species (ROS) and reactive nitrogen species (RNS), [i.e., (ROS/RNS)], and finally peroxidation. Reactive Oxygen Species (ROS) and Reactive Nitrogen Species (RNS) act together to damage cells, causing nitrosative stress. Various antioxidants have been used both experimentally and clinically in an attempt to treat burn injuries. For instance, melatonin, a scavenger of both oxygen and nitrogen-based reactants, has been recently proposed as a supportive pharmacologic agent for treating burn victims.

Radionuclide-contaminated wounds and burns promote systemic uptake of radionuclides that greatly complicate topical decontamination and triage. Radionuclide contamination also interferes with wound healing. Free radicals formed upon exposure to ionizing radiation result in a cascade of effects including, but not limited to, e.g., DNA damage, protein oxidation, lipid peroxidation that leads to apoptotic cell death, confusion of cell signaling pathways, arrest of the cell cycle, and nuclear factor kappa binding (NFkB)-related inflammation. Although the exact underlying mechanism is not well understood, inflammation from radiation exposure is increasingly recognized as a critical determinant of longer-term consequences. Inflammation with the concomitant generation of ROS/RNS, and activation of multiple signaling pathways, represents a complex process affecting multiple tissues, and contributes to the inflammatory reaction associated with wounds and burns. Suitable topical therapeutic agents that prevent systemic uptake of radionuclides from a wound or burn that can simultaneously reduce hemorrhage and severity of thermal burn injury can greatly improve patient health.

The present invention provides a new topical applicator composition and process that addresses these needs. Additional advantages and novel features of the present invention will be set forth as follows and will be readily apparent from the descriptions and demonstrations set forth herein. Accordingly, the following descriptions of the present invention should be seen as illustrative and not as limiting in any way.

### SUMMARY OF THE INVENTION

The invention includes a topical applicator composition and process for simultaneously and synergistically sequestering and removing (decorporating) radionuclides from radiologically-contaminated dermal injuries (e.g., wounds and burns). The invention provides a new radiation countermeasure that minimizes systemic absorption of radionuclides, and promotes and accelerates healing of radiologically-contaminated dermal injuries including, e.g., wounds and burns. The topical applicator composition includes a decorporation agent comprising one or more polymers (e.g., polysaccharides) mixed with an amount of a plasticizing agent that when applied to the dermal site forms a dermal covering that synergistically decorporates radionuclides, minimizes systemic absorption of radionuclides from the injury site, and promotes healing of the dermal injury. The term “plasticizing agent” as used herein means an additive introduced to the topical applicator formulation that enhances the workability, flexibility, ease of removal, or other properties of the dermal covering. In some embodiments, the plasticizing agent is a gel-forming polymer that disperses polysaccharides and/or other decorporation agents within the matrix of the polymer for removing radionuclides from the dermal surface that further enhances the ability to remove the dermal covering from the dermal surface. The term “decorporation” as used herein means “removes radionuclides from the body”. In some embodiments, the topical applicator formulation containing one or more polysaccharides and a plasticizing agent is applied directly to the dermal injury or surface in concert with, e.g., bandages, gauzes, linens, or combinations of these various approaches. In various embodiments, the topical applicator formulation further includes a selected agent that when applied to a wound or burn site controls wound promoters, including, but not limited to, e.g., bleeding, inflammation, bacterial infection, pain, or combinations of these various wound promoters. In various embodiments, the topical applicator formulations of the present invention decorporate (remove) radionuclides including, but not limited to, e.g., Co (e.g., ⁶⁰Co), Sr (e.g., ⁹⁰Sr, ⁸⁵Sr), Pu (e.g., ²³⁸Pu and ²³⁹Pu), Am (e.g., ²⁴¹Am, ^{242m}Am, and ²⁴³Am), U (e.g., ²³⁵U, ²³⁸U and depleted U), Cm (e.g. ²⁴²Cm and ²⁴⁴Cm), Cs (e.g., ¹³⁷Cs), Po (e.g., ²¹⁰Po), including combinations of these radionuclides. Exemplary polysaccharides used in conjunction with the invention include, but are not limited to, e.g., chitosan, chitin, alginic acid or its salt, hyaluronic acid or its salt, hyaluronan, fucoidin, fucoidan, carrageenan, and other non-toxic polysaccharides, including combinations of these polysaccharides. In a preferred approach, polysaccharides are naturally-derived, but are not limited thereto. As an exemplary polysaccharide, chitosan as a biomaterial is non-toxic, and exhibits anti-inflammatory and hemostatic properties which promote wound healing and attenuate biological effects of ionizing radiation. Chitosan films are also permeable to oxygen and water vapor, control bleeding, and act as an effective barrier between bacteria and open wounds. Chitosan, in combination and other polysaccharides described herein, is uniquely suited for removal of radionuclides from radionuclide-contaminated dermal wounds and burns. Preferred polysaccharides have a chemical composition defined by the formula [(W₂)ₙ—R], where: (W₂) is at least one of a C-1 to C-6 alkyl, or a C-1 to C-6 arylalkyl moiety optionally coupled to R; (R) is an amino (—NH—), carboxylic (—COOH), ester (—COO—), ether (—O—), amide (—NCO—), sulfate (—SO₃H), thiol (—S—), or hydroxy (—OH) functionality; and (n) is an integer. The plasticizing agent can include a preselected quantity of a water-soluble additive that enhances the flexibility of the dermal covering on the dermal surface or injury site. The plasticizing agent further provides for easy detachment and replacement or exchange of the topical applicator covering that minimizes pain associated with the replacement or exchange. In one embodiment, the plasticizing agent includes an effective quantity of a polyalkylene glycol (PAG). In other embodiments, the plasticizing agent is a hydrophilic polymer, e.g., a hydrogel that gels at preselected skin temperatures. In other embodiments, the plasticizing agent includes: dibutyl sebacate (DBS); dioctyl sebacate (DOS); dactyl adipate (DOA); tri-2-ethylhexyl trimellitate (TOTM), including combinations of these polymers. The plasticizing agent can also be a quaternary ammonium salt. In various embodiments, the plasticizing agent includes: benzyltributylammonium chloride (BTBAC); benzyltriethylammonium chloride (BETEC); benzyltrimethylammonium chloride (BTMAC); 3-chloro-2-hydroxy-propyl trimethylammonium chloride (Reagens-S-CFZ); tetraethylammonium chloride (TEAC); tetramethylammonium chloride (TMAC); dodecyltrimethyl ammonium chloride (DOTAC); glycidyl trimethylammonium chloride; including combinations of these compounds. In yet other embodiments, the plasticizing agent includes: cetyltrimethylammonium bromide (CETAB); dodecyltrimethylammonium bromide (DOTAB); tetrabutylammonium bromide (TBAB); tetraethylammonium bromide (TEAB); tetrapropylammonium bromide (TPAB); benzyltriethylammonium hydroxide (BETEA-OH); benzyltrirnethylarnrnoniurn hydroxide (BTMA-OH); tetrabutylammonium hydroxide (TBA-OH); tetraethylammonium hydroxide (TEA-OH); tetramethylammonium hydroxide (TMA-OH); tetrapropylammonium hydroxide (TPA-OH); allyltriphenylphosphonium bromide (TAL); benzyltriphenylphosphonium bromide (TZP); benzyltriphenylphosphonium chloride (TBC); benzyltriphenylphosphonium iodide (TBJ); 3-Bromomethyltriphenylphosphonium bromide (BTB); butyltriphenylphosphonium bromide (TBP); butyltriphenylphosphonium chloride (BTC); 2-carboxyethyltriphenylphosphonium bromide (CET); 4-carboxybutyltriphenylphosphonium bromide (CBT); ethyltriphenylphosphonium bromide (TEP); ethyltriphenylphosphonium chloride (ETC); ethyltriphenylphosphonium iodide; formylmethyltriphenylphosphonium chloride (FMC); heptyltriphenylphosphonium bromide (TTP); hexyltriphenylphosphonium bromide (THP); isoamyltriphenylphosphonium bromide (ITB); isobutyltriphenylphosphonium bromide (TIP); methoxymethyltriphenylphosphonium chloride (MMC); methyltriphenylphosphonium bromide (TMP); methyltriphenylphosphonium iodide (MPJ); pentyltriphenylphosphonium bromide (TPL); propyltriphenylphosphonium bromide (TPP); tetraphenylphosphonium bromide (TTB); tetraphenylphosphoniurn iodide; and combinations of these compounds.

The composition can further include one or more synthetic decorporation (sequestration and removal) agents that enhance the efficacy of the composition toward decorporation of radionuclides present in a radiologically-contaminated dermal surface, including, e.g., a dermal injury site such as a wound or a burn. In various embodiments, decorporation agents include, e.g., diethylene triamine pentaacetic acid (DTPA); ethylenediamine-tetraacetate (EDTA); 1-hydroxy-2(1H)-pyridinone-based octadentate ligands (HOPO), including, e.g., 1,2-HOPO and Me-3,2-HOPO metal chelating units; D-penicillamine (DPA); 2,3-dimercaptopropanol (BAL); meso-2,3-dimercaptosuccinic acid (DMSA), sodium 2,3-dimercaptopropane-1-sulfonate (DMPS); N,N′-bis(2-aminoethyl)-1,2-ethanediamine dihydrochloride (trientine), Prussian Blue, SAMMS™ sorbents composed of self-assembled monolayers on mesoporous supports, derivatives thereof, including combinations of these various decorporation agents. In various embodiments, SAMMS sorbents include, but are not limited to, e.g., acetamide phosphonic acid (AcPhos)-SAMMS; thiol (SH)-SAMMS, iminodiacetic acid (IDAA)-SAMMS; glycinyl-urea (Gly-Ur)-SAMMS; and ferrocyanide-ethylenediamine (FC-EDA)-SAMMS that further contain a transition metal (e.g., copper).

In some embodiments, the topical applicator composition includes: about 0.01% to about 5% by weight of polyalkylene glycol, about 1.0% to 5.0% by weight of each of at least three members selected from: chitosan, chitin, hyaluronan, alginate, focoidin; and from about 0.01% to 5% by weight of one or more of: calcium, sodium, potassium, magnesium, chloride, and phosphate mixed with a sufficient quantity of a plasticizing reagent.

In other embodiments, the topical applicator composition includes about 1.0% to 5.0% by weight of each of at least three members selected from: chitosan, chitin, hyaluronan, alginate, focoidin; and from about 1% to about 10% by weight of one or more SAMMS sorbents mixed with a sufficient quantity of a hydrophilic gel-forming polymer as the plasticizing agent.

In yet other embodiments, the topical applicator composition further includes an adhesive agent or component to adhere the applicator to the dermal wound. In some embodiments, the topical applicator can include an inflammation reducing agent, an absorption agent or component to absorb exudates, an antimicrobial agent, a steroidal agent (e.g., a corticosteroid), including combinations of these various agents to promote healing and prevent infection. In a preferred embodiment, the topical applicator composition is applied directly to the dermal surface. In other embodiments, the topical applicator includes a bandage or gauze. In various embodiments, the dermal applicator is replaced or exchanged following decorporation of radionuclides to reduce the radiological burden from the dermal injury or wound site and to further minimize potential for systemic absorption.

The purpose of the foregoing abstract is to enable the United States Patent and Trademark Office and the public generally, especially scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Various advantages and novel features of the present invention are described herein and will become further readily apparent to those skilled in this art from the following detailed description. In the preceding and following descriptions the preferred embodiment of the invention is shown and described by way of illustration of the best mode contemplated for carrying out the invention. As will be realized, the invention is capable of modification in various respects without departing from the invention. Thus, there is no intention to limit the invention to the specific form disclosed, but, on the contrary, the invention is intended to cover all modifications, alternative constructions, and equivalents falling within the scope of the invention as defined in the claims. Accordingly, the drawings and description of the preferred embodiment set forth hereafter are to be regarded as illustrative in nature, and not as restrictive.

A more complete appreciation of the invention will be readily obtained by reference to the following description of the accompanying drawings in which like numerals in different figures represent the same structures or elements.

### BRIEF DESCRIPTION OF THE DRAWINGS

FIG. 1 shows representative polysaccharides suitable for use in conjunction with the invention.

FIG. 2 shows one embodiment of the invention.

FIG. 3 shows an exemplary process for decorporation of radionuclides from a radiologically-contaminated dermal injury, in accordance with the invention.

FIG. 4 presents UV-VIS measurements for a solution containing Co (II) and chitosan oligosaccharide lactate.

FIGS. 5a-5b compares urinary and fecal elimination of ⁶⁰Co administered orally in rats.

FIG. 6 compares urinary elimination of ⁸⁵Sr, and concentration of ⁸⁵Sr in the femur bone in rats treated with alginate.

FIG. 7 shows blood concentrations of ¹³⁷Cs as a function of time following dosing of a dermal surface.

### DETAILED DESCRIPTION

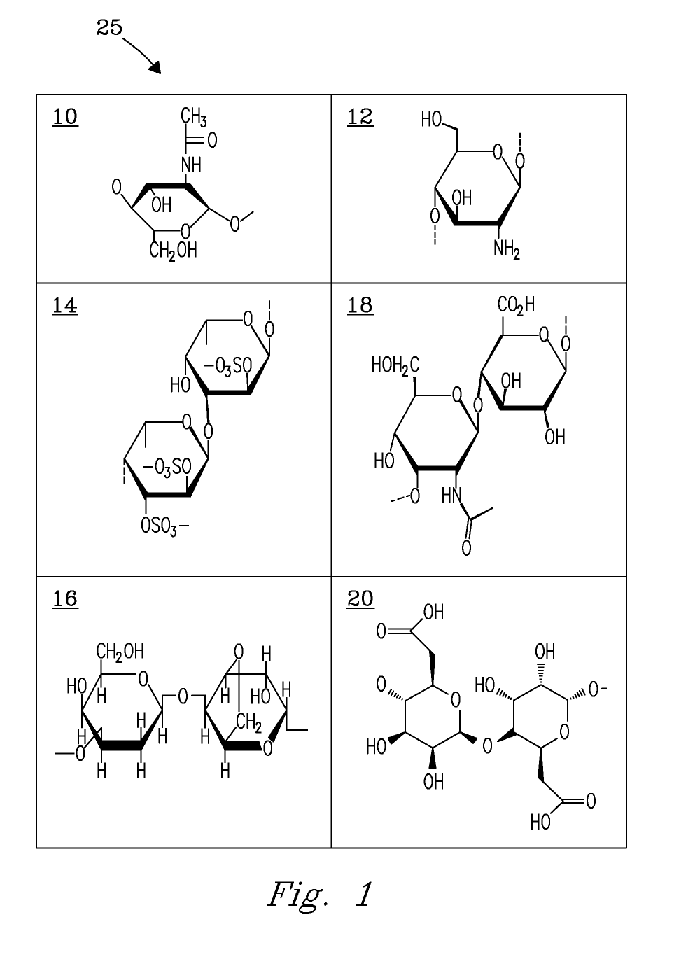

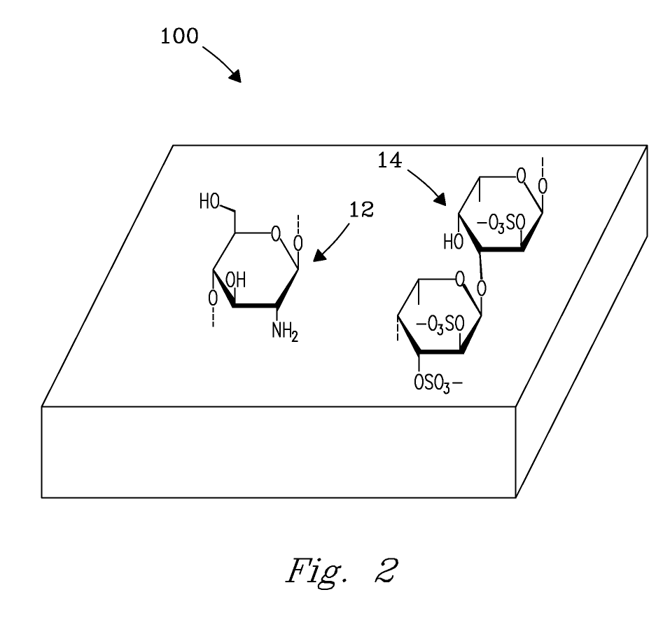

A polysaccharide-based topical applicator and process are disclosed that decorporate radionuclides and promote healing of radiologically-contaminated dermal injuries. The term “dermal injury” as used herein means any radiologically-contaminated wound, sore, ulcer, or burn of the skin and underlying dermal layers. In various embodiments, topical applicators of the present invention containing various mixed polysaccharides and other agents provide effective and non-toxic localized treatment that can simultaneously remove radionuclides and promote or accelerate healing from radiologically-contaminated dermal surface injuries. The topical applicators can be directly used as a medical countermeasure in the event of radiological and nuclear threats, and further provides a post-exposure treatment regimen that mitigates effects of internal radionuclide contamination. The invention finds use, e.g., in biomedical applications. FIG. 1 shows exemplary decorporation agents 10 suitable for use in conjunction with the invention. Decorporation agents 10 are composed of one or more polysaccharides 25 that define decorporation agents 10. Polysaccharides 25 include, but are not limited to, e.g., chitin 10 and derivatives thereof, chitosan 12, fucoidan (fucoidin) 14, carrageenan 16, hyaluronic acid and its salts 18 (e.g., hyaluronan), alginic acid and its salts (e.g., alginate) 20, and other non-toxic polysaccharides, including combinations of these polysaccharides. Topical applicators of the invention (described further in reference to FIG. 2) can include one or more of these polysaccharides 25 as decorporation agents 25, and other constituents that promote wound healing (described further herein). When deployed on a dermal injury, these polysaccharides 25 decorporate radionuclides from radiologically-contaminated dermal injuries and promote healing. Chitosan 12, for example, is a natural cationic polymer prepared by N-deacetylation of chitin 10, and is biodegradable and non-toxic. Chitosan 12 controls bleeding and provides an effective barrier between an open wound and bacteria. In gel form, chitosan 12 acts as an ideal wound dressing. It is biocompatible, biodegradable, hemostatic, anti-infective and accelerates wound healing. Hemostatic activity of chitosan wound dressings is attributed, in part, to the positively charged chitosan molecules that attract negatively charged red blood cells and platelets. Red blood cells fuse with the chitosan dressing to form a tight clot on the surface of the wound. Chitosan 12 and alginate 20 are both non-toxic agents and exhibit anti-inflammatory and hemostatic activities. These agents promote wound healing and attenuate various biological effects of ionizing radiation. Chitosan films are also permeable to oxygen and water vapor, control bleeding, and act as an effective barrier to entry of bacteria in an open wound. Thus, dressing a skin burn or wound with a chitosan-based topical applicator 100 of the invention can hinder pathogenesis mechanisms and promote recovery of a radiologically-contaminated dermal injury. Chitosan-based topical applicators can further prevent systemic absorption of radioisotopes in radiologically-contaminated wounds and burns, e.g., CRCI, detailed hereafter.

### Preventing Systemic Absorption of Radionuclides

In some embodiments, chitosan 12 and alginate 20 find application for removing radionuclides from radiologically-contaminated wounds and burns that may prevent systemic absorption of the radionuclides, a benefit in addition to their ability to promote healing of dermal injuries. Chitin 10 and chitosan 12 have been shown to block systemic absorption of radionuclides ²⁴¹Am and ⁶⁰Co from the GI tract. And, chitosan 12 is also effective for decorporation of intravenously administered ²³³U and ingested ⁶⁰Co. In some embodiments, chitin 10 and alginate 20 can be used to prevent systemic uptake of ingested ²⁴¹Am and ⁸⁵Sr, respectively. In some embodiments, alginate 20, which is highly absorbent and biodegradable, can be employed in wound dressings, and can also be applied to cleanse a wide variety of secreting lesions, including, e.g., ulcers. The calcium salt of alginate 20 (calcium alginate) increases the proliferation of fibroblasts and thus improve some cellular aspects of normal wound healing. In some embodiments, sodium alginate 20, the sodium salt of alginic acid, can be used to suppress strontium (Sr) absorption from the gastrointestinal tract without interference with calcium. Thus, chitosan 12 and alginate 20, including various salts, either alone or in combination, are uniquely suited for treatment of wounds and burns contaminated with radionuclides. In other embodiments, fucoidan 14, another polysaccharide obtained from brown algae can be used. Fucoidan 14 has an effect on inflammation, cell proliferation, and cell adhesion. In some embodiments, wound healing is synergistically accelerated by combining chitosan 12 and fucoidan 14 topical films to manifest rapid dermal papillary formation, re-epithelization, and wound closure. Results are attributed to a high affinity by fucoidan 14 for fibroblasts and an increased applicator efficacy (described further in reference to FIG. 2) due to binding with cytokines and other factors important for wound healing. In other embodiments, hyaluronic acid (HA) 18, another polysaccharide possessing beneficial properties for wound healing is employed for decorporation and treatment of radiologically-contaminated wounds and burns. Hyaluronic acid (HA) is produced by fibroblasts and other specialized connective tissue cells. HA 18 is distributed widely throughout connective, epithelial, and live neural tissues. As such, HA 18 is a chief component of the extracellular matrix, and contributes significantly to cell proliferation and migration. It is also a major component of skin and is involved in tissue repair. Thus, in various embodiments, hyaluronic acid can be included with polysaccharide topical applicators of the invention (described further in reference to FIG. 2) as an effective healing agent. In various embodiments, healing benefits of various polysaccharides 25 can be further enhanced by combining the polysaccharides with other synergistic, wound-healing promoting components, described further herein. Polysaccharides 25 used in conjunction with the invention are commercially and readily available and can also be easily produced in large quantities. Further, the non-toxic nature of such agents, along with their current use in the medical and pharmaceutical fields allows these agents to be safely and quickly distributed to the general public for medical mitigation applications.

FIG. 2 shows an exemplary topical applicator 100 of the invention of a bandage or gauze pad design. Topical applicator 100 includes a decorporation agent comprising one or more polysaccharides. In the figure, topical applicator 100 is shown with two polysaccharides, i.e., chitosan 12 and fucoidan 14, but is not limited thereto. Polysaccharides (FIG. 1) include, but are not limited to, e.g., chitin and derivatives thereof, chitosan, alginic acid and its salts (e.g., alginate), hyaluronic acid and its salts, hyaluronan, fucoidan, fucoidin, carrageenan, other non-toxic polysaccharides, derivatives thereof, and combinations of these various polysaccharides. Topical applicators 100 of the invention that apply polysaccharides described herein can synergistically enhance decorporation of various radionuclides from radiologically-contaminated dermal wounds and burns. In particular, the invention provides an optimal treatment regimen for reducing the radioactive burden associated with exposure to radionuclides that can potentially enter the body through the wound or burn site. Other wound healing constituents can also be included that promote healing efficacy to these contaminated dermal wounds and burns contaminated with radionuclides. In various embodiments, topical applicator 100 can include: 1) a decorporation agent comprising one or more polysaccharides that removes radionuclides from a dermal injury; 2) a plasticizing agent for mixing the polysaccharides and sealing the polysaccharides within the dermal matrix of the topical applicator; 3) drugs; 4) anti-inflammatory agents; 5) hemostatic agents; 6) antimicrobial agents; 7) pain control agents; 8) chelating agents; 9) steroid agents (e.g., corticosteroids); 10) absorbent materials including, but not limited to, e.g., cloth, sponges, fabrics, and like materials for removing exudates from wounds and burn sites; including combinations of these various agents. In concert with these selected agents, topical applicators 100 of the invention can be more effective than single polysaccharide-based bandages in controlling bleeding, reducing inflammation, reducing pain, providing an effective barrier in open wounds against infection, and providing properties that promote wound healing. The topical applicator 100 composition of the invention is preferably applied directly to a dermal injury site. However, in some embodiments, the topical applicator 100 composition may also be applied to a dermal injury in conjunction with, e.g., bandages and gauzes. When applied to a wound or burn site, the topical applicator composition controls wound promoters including, but not limited to, e.g., bleeding, inflammation, bacterial infection, pain, and combinations of these promoters. Topical applicators 100 can include any shape, e.g., square, rectangular, triangular, round. Thus, no limitations are intended.

### Preparation of Polysaccharide Topical Applicator Thin Films/Gels

High-viscosity (>400 mPa as 1% solution in acetic acid at 20° C.) chitosan from crab shells (≦1% insoluble matter), fucoidan from Fucus vesiculosus, alginic acid sodium salt from Brown Algae (alginate), and hyaluronic acid sodium salt from Streptococcus equi (hyaluronate) are commercially available (Sigma-Aldrich, St. Louis, Mo., USA). In an exemplary process for preparation of chitosan-based or mixed polysaccharide-based topical applicator films, a weighted amount of high-viscosity chitosan, fucoidan, alginate, or hyaluronate can be used. Weighted amounts of the polysaccharide (alone or in combination) can be added to a 0.5% to 1% acetic acid or a lactic acid solution, which is then stirred (e.g., with a magnetic stirrer overnight at room temperature) to form a clear pale yellow solution containing between about 2% and 4% chitosan. In some embodiments, an aqueous solution of propylene glycol, glycerol, or another plasticizer can be added as a plasticizer agent. Solution pH can be adjusted with NaOH as needed to attain a pH between 5.3 and 5.5 (the pH of human skin), or another desired pH. The resulting solution can be degassed, e.g., by sonication, and dried to form a gel-like film. In some embodiments, chitosan-based gel-like films for topical application in conjunction with the invention can be prepared as described, e.g., by Alemdaro\check{g}lu C et al. (Burns. 2006, 32:319-327) and Jackson (U.S. Pat. No. 4,659,700A. 1987) incorporated herein.

### Tailoring of Physicochemical Properties of Selected Polysaccharide-Based Topical Applicators

Topical applicators of the invention can include various physicochemical properties. Physicochemical properties include, but are not limited to, e.g., molecular weight, moisture content, viscosity, and degree of deacetylation. In various embodiments, the topical applicator preparation formulation includes suitable physicochemical properties (e.g., molecular weight, moisture content, viscosity, degree of deacetylation) and adhesive properties for local treatment, e.g., of thermal burns. In some embodiments, the topical applicator can be applied as a viscous gel rather than a thin film. In various embodiments, varying amounts of calcium alginate can be added to the polysaccharide formulation to provide a suitable gel viscosity and desired water content. In some embodiments, topical applicator formulations made with chitosan gels can also include alginate materials to increase moisture content and flexibility, e.g., to prevent the chitosan gels from becoming rigid in contact with a wound (e.g., due to hemostatic properties of the wound). Tailoring various physical properties of the topical applicator can be important, e.g., to provide painless and trauma-free dressing changes during treatment of thermal burns. In various embodiments, topical applicator dressings of the invention can further include a quantity of the polysaccharide calcium alginate to give the topical applicator gel-like surface properties that addresses bleeding wounds and reduces pain during dressing changes. Calcium alginate also provides a moist wound environment that promotes rapid granulation and re-epithelialization of dermal tissues. In various embodiments, topical applicators of the invention can also include mixed alginate-polysaccharides. Mixed alginates allow physical properties (e.g., flexibility) of topical gels/films to be adjusted. In some embodiments, complex chitosan-alginate membranes can also be used to accelerate healing of dermal wounds. In some embodiments, either fucoidan or HA can be added at an appropriate concentration or ratio to maintain desired properties for the topical gel. In various embodiments, topical applicators of the invention can include different plasticizers (e.g., propylene glycol, glycerol) and weak acids (e.g., acetic acid, lactic acid, glycolic acid) to provide polysaccharide films with suitable properties. In various embodiments, topical applicator gels/films can be prepared that have properties including, but not limited to, e.g., selected thicknesses, water absorption capacities, water vapor permeabilities, mechanical strength, elasticity, bioadhesion, and combinations of these various properties. In some embodiments, calcium alginate can be added to the polysaccharide formulation to adjust gel viscosity and moisture content. In other embodiments, calcium alginate-based wound dressings are applied to promote rapid granulation and re-epithelialization of dermal tissues for bleeding wounds and to reduce pain during dressing changes,

### Decorporation Agents

The topical applicator composition can include one or more decorporation agents to enhance the efficacy for removing radionuclides present in radiologically-contaminated dermal injuries (e.g., wounds and burns). Decorporation agents include, but are not limited to, e.g., DTPA, EDTA, HOPO, DPA, BAL, DMSA, trientine, Prussian Blue, SAMMS sorbents, derivatives thereof, including combinations of these various agents. In some embodiments, SAMMS™ sorbents (Steward Advanced Materials, Inc., Chattanooga, Tenn., USA) can be utilized in conjunction with the invention for decorporation of radionuclides as detailed, e.g., by Fryxell et al. in co-pending U.S. patent application Ser. No. 12/613,998 filed 6 Nov. 2009, which reference is incorporated herein in its entirety. SAMMS materials include a rigid, porous backbone that when functionalized with specific chemically-selective ligands, provides selective attachment to, and sequestration of, specific target materials. In the present invention, SAMMS sorbents can be used to decorporate radionuclides including, e.g., actinides, and other radionuclides including, e.g., ¹³⁷Cs and ²¹⁰Po. SAMMS sorbents suitable for use in conjunction with the invention include: acetamide phosphonic add (AcPhos)-SAMMS; thiol (SH)-SAMMS; iminodiacetic add (IDAA)-SAMMS; glycinyl-urea (Gly-Ur)-SAMMS; and ferrocyanide (FC-Cu-EDA)-SAMMS. SAMMS sorbents provide enhanced selectivity for targeting radionuclides than many chelating agents in terms of efficacy, convenient administration, and safe use. In the topical applicator polysaccharide composition or matrix, one or more SAMMS sorbents may be added to provide a high affinity for target radionuclide(s), rapid metal binding rates, and a large sorption capacity. Thus, SAMMS sorbents can effectively decorporate and retain toxic species thus limiting systemic absorption of radionuclides from dermal injuries and surfaces. SAMMS sorbents best suited for capture of targeted radionuclides can be selected using distribution coefficients (K_{d}, mL/g) for the selected sorbent that provide affinities for target species of interest. SAMMS sorbents can be prepared at various mesh sizes for incorporation within the selected topical applicator matrix for delivery and application on the dermal surface. The selected SAMMS sorbent (preselected grain size) can be suspended, e.g., in a transport buffer (pH 7.4) consisting of 1.98 g/L of glucose, 10% (v/v) of 10× Hank's salt solution balanced with Ca and Mg, and 0.01M of HEPES buffer [4-(2-hydroxyethyl)-1-piperazineethanesulfonic acid] at the S/L ratio of 10 g/L. This suspension can then be added to the selected polymer topical applicator and mixed with the plasticizing agent for delivery to the dermal surface directly or to a bandage or gauze that is then placed on the dermal surface. In other embodiments, the selected SAMMS sorbent can be dispersed in a selected polymer and plasticizer (e.g., a thermo-sensitive, gel-forming polymer) for delivery to the dermal surface, as discussed further herein. No limitations are intended.

### Plasticizing Agents

In various embodiments, the topical applicator composition includes one or more plasticizing agents that provide the dermal covering with a suitable plasticity, flexibility, or fluidity on the dermal wound site. In various embodiments, the plasticizing agent further allows easy and rapid detachment of the topical applicator for purposes of replacement or exchange while minimizing pain associated with the replacement or exchange from the dermal site. In some embodiments, the plasticizing agent is water. In one embodiment, the plasticizing agent includes a quantity of a polyalkylene glycol (PAG). In various other embodiments, the plasticizing agent is a hydrophilic polymer (hydrogel). Hydrophilic polymers include, e.g., dibutyl sebacate (DBS); dioctyl sebacate (DOS); doctyl adipate (DOA); tri-2-ethylhexyl trimellitate (TOTM), including combinations of these polymers. In some embodiments, the plasticizing agent can also be a quaternary ammonium salt. In various embodiments, the plasticizing agent includes: benzyltributylammonium chloride (BTBAC); Benzyltriethylammonium chloride (BETEC); benzyltrimethylammonium chloride (BTMAC); 3-chloro-2-hydroxy-propyl trimethylammonium chloride (Reagens-S-CFZ); tetraethylammonium chloride (TEAC); tetramethylammonium chloride (TMAC); dodecyltrimethyl ammonium chloride (DOTAC); glycidyl trimethylammonium chloride; including combinations of these compounds. In yet other embodiments, the plasticizing agent includes: cetyltrimethylammonium bromide (CETAB); dodecyltrimethylammonium bromide (DOTAB); tetrabutylammonium bromide (TBAB); tetraethylammonium bromide (TEAK); tetrapropylammonium bromide (TPAB); including combinations of these compounds. In some embodiments, the plasticizing agent includes: benzyltriethylammonium hydroxide (BETEA-OH); benzyltrimethylammonium hydroxide (BTMA-OH); tetrabutylammonium hydroxide (TBA-OH); tetraethylammonium hydroxide (TEA-OH); tetramethylammonium hydroxide (TMA-OH); tetrapropylammonium hydroxide (TPA-OH); and combinations of these reagents. In some embodiments, the plasticizing agent includes: allyltriphenylphosphonium bromide (TAL); benzyltriphenylphosphonium bromide (TZP); benzyltriphenylphosphonium chloride (TBC); benzyltriphenylphosphonium iodide (TBJ); 3-Bromomethyltriphenylphosphonium bromide (BTB); butyltriphenylphosphonium bromide (TBP); butyltriphenylphosphonium chloride (BTC); 2-Carboxyethyltriphenylphosphonium bromide (CET); 4-Carboxybutyltriphenylphosphonium bromide (CBT); ethyltriphenylphosphonium bromide (TEP); ethyltriphenylphosphonium chloride (ETC); ethyltriphenylphosphonium iodide; formylmethyltriphenylphosphonium chloride (FMC); heptyltriphenylphosphonium bromide (TTP); hexyltriphenylphosphonium bromide (THP); isoamyltriphenylphosphonium bromide (ITB); isobutyltriphenylphosphonium bromide (TIP); methoxymethyltriphenylphosphonium chloride (MMC); methyltriphenylphosphonium bromide (TMP); methyltriphenylphosphonium iodide (MPJ); pentyltriphenylphosphonium bromide (TPL); propyltriphenylphosphonium bromide (TPP); tetraphenylphosphonium bromide (TTB); tetraphenylphosphonium iodide; and combinations of these compounds.

### Treatment of Radiologically Contaminated Wounds and Burns

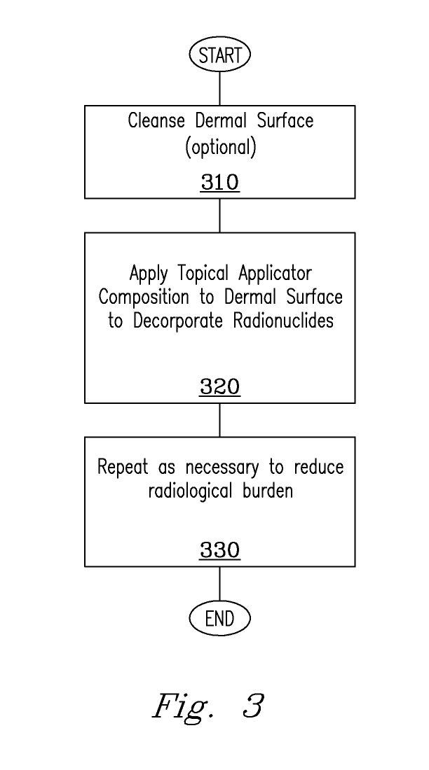

The invention on provides a mature and promising post-exposure treatment product to mitigate effects of radionuclide contaminated cutaneous radiation injuries (CRCI), e.g., wounds and burns. FIG. 3 shows exemplary process steps for treating radionuclide-contaminated wounds and burns for decorporation of radionuclides from a wound in conjunction with one embodiment of the invention. {START}. In a first (optional) triage step {310} for treatment of cutaneous radiation injury, the outer epidermis (stratum corneum) layer of the skin is cleansed to remove callus, as well as radiologically-contaminated particulates and debris. In another step {320}, a topical applicator comprising a mixture of polysaccharides and other selected wound treatment components is applied to the injury sites to synergistically enhance decorporation efficacy and promote healing. In case of extended burns or severe hemorrhage, wound decontamination can be inefficient. Thus, effects of the selected polysaccharides in the topical applicator can be enhanced by combining with other synergistic components, including, decorporation agents (e.g., chelating agents) to aide removal of specific radionuclides; wound healing agents to promote healing of the dermal injury; and antimicrobials to prevent infection. For example, silver sulphadiazine, a broad-spectrum antimicrobial, also provides additional benefits including, e.g., pain management. Formation of exemplary polysaccharide-based films (e.g., chitosan) are detailed, e.g., by Alemdaro\check{g}lu C et al. in [Burns 2006, 32:319-327] and by Jackson et al. in [U.S. Pat. No. 4,659,700A1. 1987], which references are incorporated in their entirety herein. Polysaccharides in the topical applicator can exhibit anti-inflammatory and hemostatic properties that promote healing of dermal injury wounds or burns, prevent systemic absorption of radioisotopes in the contaminated wounds, and attenuate biological effects associated with ionizing radiation released by radionuclides present in the wound. In another step, {330}, the process can be repeated as necessary to reduce the radiological burden in the dermal injury, or to reduce the likelihood of systemic migration of radionuclides from the dermal injury sites to the whole body. {END}.

### Decorporation Affinity

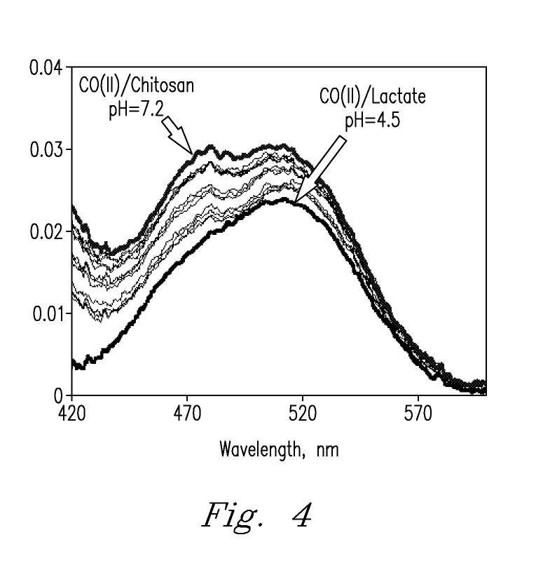

FIG. 4 shows UV-VIS data collected from an exemplary optical test using a solution containing Co (II) (3.88 mM) chloride titrated with NaOH over a pH range from about pH 4.5 to pH 7.2 in the presence of chitosan oligosaccharide lactate (1.4 wt %)—a lactate chelator competitor. The solution further contained 0.2 M NaClO₄, a non-reactive salt used to control ionic strength of the solution. The spectral layout (experimental design) for UV-VIS measurements included a 1-cm path length (Beers Law parameter for interpreting concentration from absorbance measurements), an argon atmosphere, and a temperature of about 22° C. Spectral data were fit using SQUAD computer modeling, as detailed in “Stability Quotients from Absorbance Data”, D J Leggett, editor, in“Computational Methods for the Determination of Formation Constants”, Plenum Press, New York, 1985. Results show that even in the presence of the lactate chelator competitor, chitosan strongly chelates Co(II), as evidenced by the formation of new band at 472 nm. The formation constant (K_{f}) for the complex species [Co²⁺.Chitosan₂.(OH)⁻] was determined to be −1.67±0.02 on the logarithmic scale. Chitosan similarly exhibited strong affinity for Nd(III) in vitro, a mimic for Am(III) and U(VI). In various embodiments, radiation countermeasures provided by the invention can show similar decorporation efficacy toward various radionuclides, and significantly expand currently limited options for preventing systemic absorption of radionuclides present in radiologically-contaminated dermal injuries, e.g., wounds and burns. Binding affinity of administered chelators can be assessed by comparing radionuclide or radioactivity levels in specimens treated with the topical applicator against control specimens receiving radioisotopes only. TABLE 1 lists the protocols for oral administration and exposure of F344 rats to ⁶⁰Co, and subsequent oral administration of chitosan and other polysaccharides.

TABLE 1
Animal study protocol describing oral exposure of F344 rats to ⁶⁰Co
and treatment by oral chitosan and other polysaccharides.

| | | | | | | | | | Time |
|---|---|---|---|---|---|---|---|---|---|
| | | | | | | | | | from |
| | | | ⁶⁰Co | | | | | | ⁶⁰Co |
| | No. | | Expose | | | | | | Expose |
| | of | | (Dose) | No. of | | | Chelator | Dose^{a} | to |
| Group | Rats | Route | kBq^{a} | Doses | Chelator | Route | Admin. | (mg/kg) | Sacrif. |
| 1 | 6 | Oral | 7.1 ± 0.2 | 1 | — | Oral | n/a | — | 48 |
| 2 | 6 | Oral | 12.1 ± 0.9 | 1 | — | Oral | n/a | — | 48 |
| 3 | 6 | Oral | 7.1 ± 0.2 | 1 | Chitosan | Oral | 315 ± 14 | 2 | 48 |
| 4 | 6 | Oral | 12.7 ± 1 | 1 | Chitosan | Oral | 336 ± 16 | 2 | 48 |
| 5 | 6 | Oral | 13.2 ± 0.6 | 1 | Chitosan | Oral | 288 ± 12 | 2 | 48 |
| | | | | | Lactate | | | | |
| 6 | 6 | Oral | 7.0 ± 0.2 | 1 | Alginate | Oral | 199 ± 10 | 2 | 48 |

^{a}Average ± standard deviation

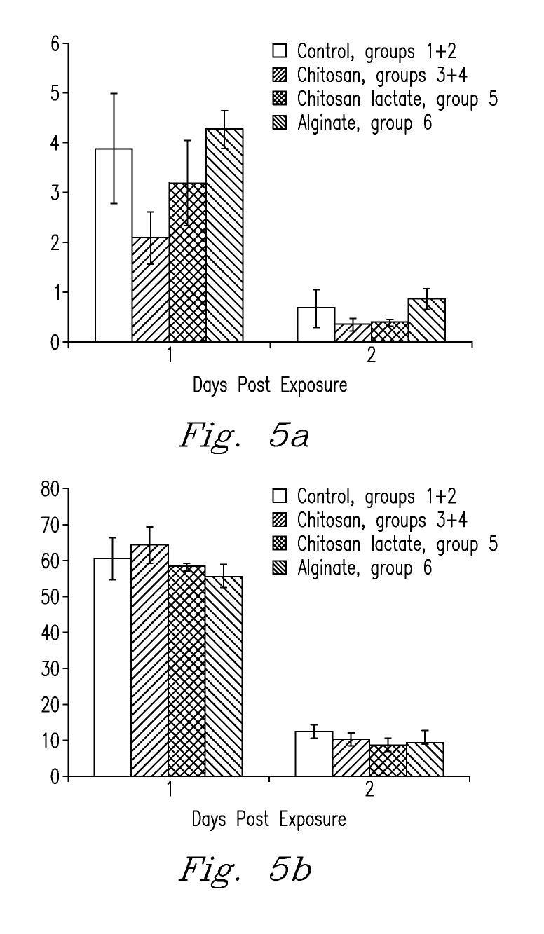

FIGS. 5a-5b show urinary (A) and fecal (B) elimination expressed as an average percent of an orally administered dose of ⁶⁰Co given to F344 rats compared with effect of oral treatment with decorporation agents. As shown in the figure, orally administered chitosan significantly suppressed systemic uptake of ⁶⁰Co from the GI tract, as evidenced by the decreased urinary excretion of ⁶⁰Co for animals in groups 3 and 4 in which oral administration of ⁶⁰Co was followed by oral administration of chitosan, as compared with animals in control groups 1 and 2 in which ⁶⁰Co was administered without subsequent treatment with chitosan. TABLE 2 lists ⁶⁰Co values in various tissues following treatment with chitosan.

TABLE 2
Tissue distribution of orally administered ⁶⁰Co in F344 rats: effect of
oral treatment with decorporation agents.

| | ⁶⁰Co % Dose, for animal group^{a} | | | | |
|---|---|---|---|---|---|
| | | | ⁶⁰Co + | | |
| | | ⁶⁰Co + | Chitosan | ⁶⁰Co + | % Reduction |
| | ⁶⁰Co Control | Chitosan | lactate | Alginate | for Chitosan |
| Tissue | Groups 1 + 2 | Groups 3 + 4 | Group 5 | Group 6 | Groups 3 + 4 |
| Kidney | 0.033 ± 0.023 | 0.014 ± 0.007^{c} | 0.020 ± 0.009 | 0.089 ± 0.063 | 58 |
| Liver | 0.27 ± 0.19 | 0.12 ± 0.06^{c} | 0.24 ± 0.20 | 0.22 ± 0.07 | 56 |
| Blood^{b} | 0.155 ± 0.058 | 0.146 ± 0.057 | 0.142 ± 0.045 | NM | 0 |
| Total skeleton^{b} | 0.098 ± 0.067 | 0.037 ± 0.022^{c} | 0.066 ± 0.054 | 0.024 ± 0.037 | 62 |
| Small Intestine | 0.053 ± 0.014 | 0.048 ± 0.007 | NM | 0.053 ± 0.018 | 0 |
| Muscle^{b} | 1.00 ± 0.55 | 1.34 ± 0.54 | 1.36 ± 0.72 | NM | 0 |

^{a}Average ± standard deviation for n = number of animals given in TABLE 1.
^{b}Calculation assumes that total blood, skeleton, or muscle is approximately 6, 7.3, or 40%, respectively, of the body weight of the animal (Brown et al. 1997).
^{c}Statistically different from corresponding control group by two tailed t-test (p < 0.05).
NM Not Measured.

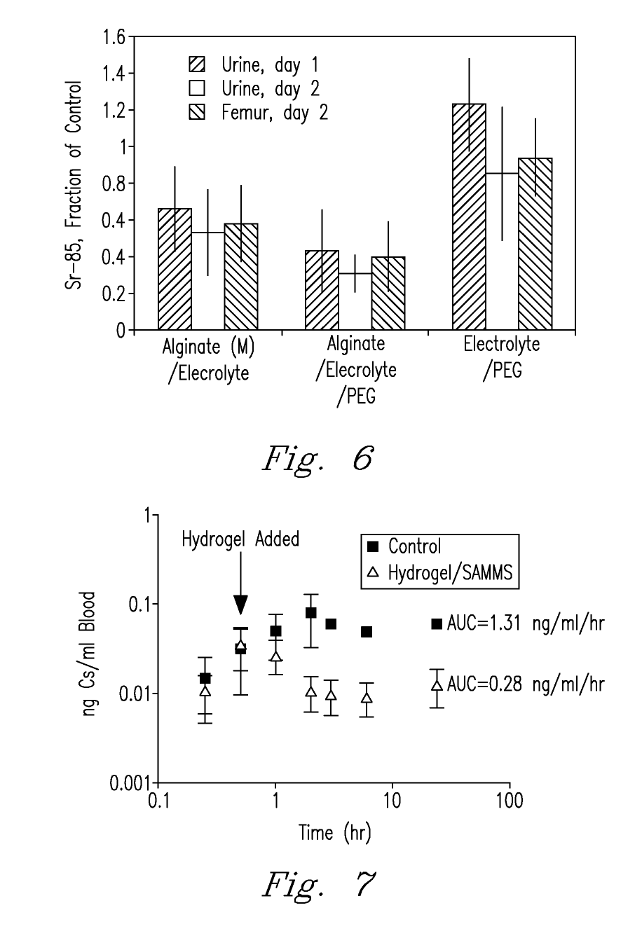

Excreta data are consistent with the observed reduction of ⁶⁰Co in tissues upon treatment with chitosan. In other embodiments, topical polysaccharide applicators can provide uptake of ⁶⁰Co and other radionuclides from contaminated wounds. FIG. 6 compares oral treatment results (2 day study) using alginate/electrolyte, alginate/electrolyte/PEG, and electrolyte/PEG on urinary elimination and concentration of ⁸⁵Sr in the femur bone, expressed as a fraction of the administered dose per gram tissue in F344 rats. Oral ⁸⁵Sr dose: 3.8 kBq; alginate: 150 mg kg⁻¹ BW. Electrolyte/PEG solution contained 60 g L⁻¹ PEG, 2.2 g L⁻¹ NaCl, and 1.7 g L⁻¹ NaHCO₃. Experimental standard deviation was between 10% and 12%. Alginate treatment suppressed systemic uptake of ⁸⁵Sr from the GI tract by 50-70%. In other embodiments, topical applicators of the invention can be used to uptake ⁹⁰Sr from radiologically-contaminated wounds.

### Radionuclide Decorporation

In various embodiments, topical applicators of the invention composed of mixed polysaccharide chelation materials can provide: 1) multi-radionuclide decorporation, and 2) significantly increase radionuclide decorporation, when compared with conventional decorporation agents, from wounds/burns in the event of a nuclear/radiological emergency. For example, in various embodiments, the present invention can be deployed to decorporate radionuclides including, e.g., ²³⁸Pu/²⁴²Pu, ²⁴¹Am, ²³³U, ⁶⁰Co, ¹³⁷ Cs, and ⁸⁵Sr. In various embodiments, radionuclides in their common stable oxidation states (which are also most common oxidation states for radionuclides in vivo) can be decorporated including, e.g., Pu(IV), Am(III), U(VI), Co(II), CO), and Sr(II).

### Measurement and Assessment of Radionuclides

To measure gamma-emitting radionuclides, dressings can be counted directly using a gamma counter. Alpha-emitting radionuclides can be removed from wound dressings by exposing the dressings to nitric acid at elevated temperatures. The radionuclides can then be measured by liquid scintillation counting (LSC). Based on the determined radioactivity, percent of administered dose for each radionuclide can be calculated. For example, tissues collected from animals exposed to a radionuclide cocktail, can be measured for gamma emitting radionuclides using either a modified protocol that allows discrimination of multiple energy windows for each particular radioisotope or by a high resolution Ge detector (Princeton Gamma-Tech IGP-1013 Model 872). Alpha-emitting radionuclides (including, e.g., ²⁴²Pu and ²³²U) can be measured in digested tissues using appropriately adjusted counting windows on liquid scintillation counting (LSC) instruments, or can be measured using a high resolution alpha spectrometer that allows simultaneous detection of multiple alpha isotopes in the same sample. Degree of binding affinity of administered chelators can be assessed by comparing radionuclide or radioactivity levels in specimens treated with the topical applicator against control specimens receiving radioisotopes only.

### Determination of Systemic Absorption of Radionuclides from Contaminated Dermal Surfaces

To determine systemic location of radionuclides, tissues (e.g., blood, bones (e.g., whole femur), brain, lung, heart, liver, spleen, kidney) can be collected and analyzed for radioactivity. Kinetics for systemic absorption of each radionuclide from a burn wound can then be determined. In one example, systemic absorption of ²³³U from contaminated wounds can be determined. In some embodiments, bandages or gauzes applied to radiologically-contaminated burns containing compositions of the present invention including one or more polysaccharides can be removed when changing dressings and tested for specific radionuclides including, e.g., ²³³U. ²³³U can be removed from the bandages by exposing the bandages to nitric acid at elevated temperatures. The acid phase containing ²³³U can then be analyzed by liquid scintillation counting (LSC). In combination, excreta (e.g., urine) can be collected daily or periodically from animals and analyzed for ²³³U by LSC. Tissues (e.g., burn wounds with surrounding skin and muscle tissue) can also be extracted, digested, and analyzed for ²³³U.

### Topical Applicators Including Antibacterial, Healing, and Chelator Agents

In some embodiments, topical applicator formulations of the present invention are applied in combination with physical dressings that further include addition of topical antibacterial agents used for treatment of burns including, e.g., creams and aqueous suspensions including, e.g., 1%-10% silver sulfadiazine. In other embodiments, topical applicators of the invention can further include agents that reduce hemorrhaging, provide effective barriers to bacterial infection, and reduce inflammatory response in the burn area. In some embodiments, topical applicators of the present invention can be prepared with one or more natural polysaccharides containing a maximum amount of each component based on solubility including, e.g., chitin, chitosan, alginate, fucoidan and hyaluronic acid (HA). In some embodiments, topical applicators can be further modified to include addition of synthetic chelators including, e.g., diethylenetriaminepentaacetic acid (DTPA) to enhance removal of radionuclides from a dermal wound site.

### SAMMS Sorbents+Gel-Forming Polymers

Decorporation of radionuclides from radiologically-contaminated dermal surfaces (including, e.g., wounds and burns) can be enhanced by addition of a SAMMS sorbent to the topical applicator formulations of the present invention. In various embodiments, various SAMMS sorbents can be delivered in a polymer, plasticizer, carrier, or other medium that contains the particles and is sufficiently hydrated to promote diffusion and uptake of radionuclides into the SAMMs nanopores, and can be easily removed from the skin, carrying the radionuclide burden with it. In various embodiments, the carrier can be a thermoreversible hydrogel. In some embodiments, the hydrogel is composed of poly(N-isopropylacrylamide) (PNIPA). Preferred hydrogel polymers are water soluble and exist in extended sol states at low temperature and undergo reversible gelation transitions at higher temperatures. Gelation temperatures can be tailored by systematically varying the composition of the polymers. Water soluble gels are advantageous in that drugs and colloidal agents can be easily incorporated into the low viscosity fluids at room temperature which are then trapped within the viscous gels upon exposure to a higher temperature physiological environment (e.g., skin). These polymers gel at concentrations as low as 10 wt %, promoting a porous, hydrated environment that allows for easy diffusion of dissolved species. An added benefit is that sols of thermoreversible polymers can be formed at room temperature and stored indefinitely before delivering them to the dermal surface which raises the temperature and forms the gel. In various embodiments, integrating the SAMMS sorbent in the sol form of the polymer permits the sol formulation to be “painted” onto a contaminated dermal surface (e.g., skin or skin wounds). As the sol warms on the patient's skin, the polymer gels, adhering the SAMMS sorbent particles in place in a hydrated matrix on the dermal surface. The open structure of the hydrogel then avows unimpeded and facile diffusion of dissolved species including, e.g., dissolved radionuclides (e.g. Cs, iodine, etc.). Radionuclides freely move throughout the SAMMS-hydrogel matrix and are captured by the SAMMS sorbent. In some embodiments, colloidal radionuclides can also be physically entrained in the hydrogel matrix. In various embodiments, after a suitable period of time, the gelled hydrogel can be peeled off the skin, removing both dissolved radionuclides and colloidal radionuclides. The radionuclides are contained in the gel phase which provides easy handling and a minimization of the risk associated with secondary dispersal or contamination. In some embodiments, a ferrocyanide-copper-ethylenediamine SAMMS (FC-Cu-EDA SAMMS) sorbent is employed. In an exemplary test, a FC-Cu-EDA SAMMS sorbent was prepared. BET surface area analysis showed the sorbent contained a specific surface area of 47 m²/g, and a pore volume of 0.43 cc/g. Cu content was 0.38 mmole Cu per gram of sorbent. Ferrocyanide (FC) content was 0.35 mmole per gram of sorbent. Next, a poly(N-isopropylacrylamide) (PNIPA) homopolymer was synthesized by placing N-isopropylacrylamide (NIPA) into dioxane, purging for 30 minutes with deoxygenated nitrogen, and polymerizing under nitrogen at 70° C. for 18 hours using 2,2′-Azobisisobutyronitrile (AIBN) (Sigma-Aldrich, St. Louis, Mo., USA) as the initiator. The polymer was cooled, diluted with acetone, and precipitated with diethyl ether. The precipitated polymer was collected by filtration, washed and dried under vacuum. The dried polymer was then dissolved in water, filtered at 0.45 μm, and further purified in ultra-filtration cells (Amicon, Inc., Beverly, Mass., USA) using a 30 kD molecular weight cutoff. To form the SAMMS/hydrogel composite, NIPAAM polymer solutions were formed at a concentration of 10 wt % to 20 wt % in water or phosphate-buffered saline (PBS) (0.15 M NaCl, 0.01 M phosphate in ultrapure Milli-Q water, at a pH of 7.4) by rotary mixing at 4° C. Suspensions of SAMMS in PBS were formed and then added to the polymer solutions at 1-5 wt % concentrations with vortexing. Rats (e.g., male Sprague-Dawley, 275 g to 325 g) with jugular vein cannulae were purchased (Charles River Laboratories, Inc., Wilmington Mass., USA). Rats were anesthetized with isoflurane and shaved to expose a dermal surface (dosing zone) on the backs of the animals. A dosing frame (5 cm×5 cm O.D., 2 cm×2 cm I.D.) was then glued to the back. The dosing zone was abraded (˜20×) using the tip of a needle to enhance dermal absorption through the skin. A dosing solution of ¹³⁷Cs chloride was prepared in aqueous buffer (pH 8.64) at a Cs concentration of 10 μg/mL and a radioactivity count of 7585 kBq/mL. The exposed skin of each rat was dosed with 25 μL of ¹³⁷Cs chloride (0.25 μg ¹³⁷Cs and 190 kBq/rat) while under anesthesia and the ¹³⁷Cs was maintained on the skin for ˜30 min. Rats in Group I (controls) received only ¹³⁷Cs chloride by dermal application to establish the dermal bioavailability and clearance rate for ¹³⁷Cs. Rats in Group II received the same dermal dose of ¹³⁷Cs, but the skin over the ¹³⁷Cs dose area was subsequently treated with 50 μL of the SAMMS/hydrogel mixture ˜30 min post ¹³⁷Cs exposure. Animals in both treatment groups were then placed under a heat lamp for an additional 30 min. The SAMMS/hydrogel covering was then removed (Group II only) by gently peeling from the skin. The covering was then subjected to gamma counting in a shielded, well-type gamma counter (e.g., Wallac 1480 WIZARD®, PerkinElmer, Waltham, Mass.). Blood (˜0.1 ml) for both treatment groups was collected through the jugular vein cannula at 0.25, 0.5, 1, 2, 3, 6 and 24 hr post-dosing (¹³⁷Cs). Excreta (urine & feces) were also collected in the metabolism cages through 24 hours post-dosing and animals were then euthanized at 24 hours. Samples were collected following euthanization for: dosed skin, bandage wrap/tape/parafilm, gel frame with attached skin, terminal blood (0.2 mL) and remaining carcass. All samples were analyzed for ¹³⁷Cs using a gamma counter. FIG. 7 plots ¹³⁷Cs concentrations in blood for rats in control group I and the FC-Cu-EDA SAMMS/hydrogel treatment group II as a function of time following dosing of the dermal surface. Groups I and II were treated the same through 30 min post dosing, which is reflected by the comparable early (0.25-1 hr)¹³⁷Cs blood kinetics between treatment groups. ¹³⁷Cs was rapidly absorbed through the abraded skin, as evidenced by the peak blood ¹³⁷Cs concentrations (0.08 ng/ml) attained within 2 hours of post-dermal exposure of rats in control Group I. Results for hydrogel/SAMMS treatment in Group II animals (beginning at 30 minutes post-dosing which remained on the skin until 1 hr post-dosing) resulted a decline in blood ¹³⁷Cs concentration (>1 hr) attributed to capture and removal of ¹³⁷Cs from the skin surface. Results show the hydrogel/SAMMS (Group II) animals absorbed nearly an order of magnitude lower ¹³⁷Cs than did the control (Group I) animals. Time-course profiles for both groups was nearly parallel. Area under the concentration (AUC) curves was 1.31 ng/ml/hr for the control group (Group I), and 0.28 ng/ml/hr for the hydrogel/SAMMS group (Group II), respectively. AUC ratios indicate nearly 80% of the ¹³⁷Cs originally applied to the skin was retained by the hydrogel/SAMMS. Control group results further show that ¹³⁷Cs blood concentration increased rapidly over the first 2 hours following dosing, reaching a peak at ˜3700 DPM/ml. After 2 hours, concentration of ¹³⁷Cs in the blood dropped to ˜2500 DPM/ml (approximately ⅔ of the peak value), achieving a quasi-steady state level which was approximately maintained for the remainder of the 24 hour study period. When the SAMMS/hydrogel was applied 30 minutes after dosing, blood concentration of ¹³⁷Cs dropped rapidly from ˜1600 DPM/ml to ˜500 DPM/ml. During this same timeframe i.e., from 30 minutes post dose to 2 hours), blood concentration for the control group rose from ˜1600 DPM/ml to ˜3700 DPM/ml, suggesting that over half of the ¹³⁷Cs dose remained on the skin during this timeframe before being absorbed. Results show that SAMMS/hydrogel formula prevented absorption into the test animals bodies. TABLE 3 lists mass balance results for ¹³⁷Cs in test animals.

TABLE 3
¹³⁷Cs concentrations in various tissues of rats from a control group
and a FC-Cu-EDA SAMMS/hydrogel group following dosing of the dermal surface.

| | % of Recovered Dose^{a,b,c} | | | | | |
|---|---|---|---|---|---|---|
| Treatment | | | | | | SAMMS/ |
| Groups | Final Blood | Skin | Urine | Feces | Carcass | Hydrogel |
| Group I | 0.06 ± 0.03 | 0.71 ± 0.15 | 7.92 ± 3.04 | 1.27 ± 0.40 | 87.2 ± 2.4 | — |
| Group II | 0.04 ± 0.03 | 1.76 ± 1.50 | 1.93 ± 1.52 | 0.18 ± 0.1 | 19.0 ± 16.3 | 76.3 ± 17.6 |

^{a}Bandages for Groups I and II had doses of 1.17 ± 0.55 and 0.42 ± 0.55 percent (%) of the recovered dose values, respectively.
^{b}Gel Frames for Groups I and II had doses of 1.44 ± 1.03 and 0.34 ± 0.19 percent (%) of the recovered dose values, respectively.
^{c}Urine wash for Groups I and II had doses of 0.26 ± 0.11 and 0.05 ± 0.03 percent (%) of the recovered dose, respectively.

Overall, mass balance results indicate that virtually all of the ¹³⁷Cs was absorbed in the control group (Group I). In this group, approximately 8% of the ¹³⁷Cs dose was excreted in the urine, and 1.3% of the ¹³⁷Cs dose was eliminated in the feces over a 24 hour period. Approximately 88% of the dose remained in the carcass after 24 hours. Results for the SAMMS/hydrogel treatment group (Group II) showed rate of absorption of ¹³⁷Cs was identical to that of the control group before the SAMMS/hydrogel formula was applied. After SAMMS/hydrogel treatment, ˜500 DPM/ml of ¹³⁷Cs remained in the blood. A steady-state level was maintained over the remainder of the 24 hour study period (corresponding to ˜13% of the original ¹³⁷Cs dose)—attributed to ¹³⁷Cs that is chemically unavailable to the SAMMS/hydrogel sorbent. A similar plateau was observed for the control group at a significantly greater ¹³⁷Cs concentration (˜2500 DPM/ml). Carcasses of the SAMMS/hydrogel treated animals had 80% lower ¹³⁷Cs compared with the control group; only ˜20% of the ¹³⁷Cs remained in the carcasses of these test animals compared with the control group. Additional time and/or repeated contact with the SAMMS/hydrogel sorbent formulations can be expected to provide enhanced retrieval of radionuclides from dermal surfaces.

While preferred embodiments of the present invention have been shown and described, it will be apparent to those of ordinary skill in the art that many changes and modifications may be made without departing from the invention in its true scope and broader aspects. The appended claims are therefore intended to cover all such changes and modifications as fall within the spirit and scope of the invention.

What is claimed:

1. A topical applicator composition for decorporation of radionuclides from a radiologically-contaminated dermal surface, comprising:
one or more polysaccharides mixed with a plasticizing agent that yields a dermal covering when applied to said dermal surface, said one or more polysaccharides in said dermal covering remove at least one radionuclide from said dermal surface.

2. The topical applicator composition of claim 1, wherein the polysaccharides are naturally-derived polysaccharides selected from the group consisting of: chitosan, chitin, alginic acid, hyaluronic acid, fucoidan, carrageenan, salts thereof, and combinations thereof.

3. The topical applicator composition of claim 1, wherein the polysaccharides have chemical formula (W₂)ₙ—R, wherein:
a. W₂ is at least one of a C-1 to C-6 alkyl, or a C-1 to C-6 arylalkyl optionally coupled to R;
b. R is an amino (—NH—), carboxylic (—COOH), ester (—COO—), ether (—O—), amide (—NCO—), sulfate (—SO3H), thiol (—S—), or hydroxy (—OH) functionality; and
c. n is an integer.

4. The topical applicator composition of claim 1, wherein the plasticizing agent includes a hydrophilic polymer dissolved in water that forms a hydrogel at a preselected skin temperature.

5. The topical applicator composition of claim 1, wherein the plasticizing agent includes a quantity of a polyalkylene glycol (PAG).

6. The topical applicator composition of claim 1, wherein the plasticizing agent is a quaternary ammonium salt.

7. The topical applicator composition of claim 1, wherein the plasticizing agent includes a water-soluble additive that enhances flexibility of the dermal covering on the dermal surface compared with the plasticizing agent absent the water-soluble additive.

8. The topical applicator composition of claim 1, further including a member selected from the group consisting of: DTPA, EDTA, HOPO, DPA, BAL, DMSA, trientine, Prussian Blue, derivatives thereof, and combinations thereof.

9. The topical applicator composition of claim 1, further including a quantity of a SAMMS sorbent mixed with a gel-forming polymer.

10. The topical applicator composition of claim 1, wherein the radionuclides removed from said dermal surface are selected from the group consisting of: Co, Sr, U, Pu, Am, Cm, Cs, Po, I, ions thereof, and combinations thereof.

11. The topical applicator composition of claim 1, comprising:
a). 1.0% to 5.0% by weight of each of at least three members selected from the group consisting of: chitosan, chitin, hyaluronan, alginate, focoidin, and combinations thereof;
b). 0.01% to 5% by weight of a polyalkylene glycol; and
c). 0.01% to 5% by weight of one or more of: calcium, sodium, potassium, magnesium, chloride, and phosphate.

12. The topical applicator composition of claim 1, including a member selected from the group consisting of: a wound healing agent; an antimicrobial agent; a hemostatic agent, a steroidal agent, an anti-inflammatory agent, a pain reduction agent, and combinations thereof.

13. The topical applicator composition of claim 1, including an adhesive agent that adheres said composition to the dermal surface.

14. The topical applicator composition of claim 1, including an absorbing agent that absorbs exudates from the dermal surface.

15. A method for removing radionuclides from a radiologically-contaminated dermal surface, comprising the steps of:
applying a dermal covering to said dermal surface, said dermal covering including one or more polymers mixed with a plasticizing agent; and
removing at least one radionuclide with said dermal covering from said dermal surface.

16. The method of claim 15, wherein the one or more polymers are polysaccharides selected from the group consisting of: chitosan, chitin, hyaluronan, alginate, focoidin, salts thereof, and combinations thereof.

17. The method of claim 15, wherein the one or more polymers are water soluble polymers.

18. The method of claim 15, wherein the one or more polymers are hydrogel-forming polymers.

19. The method of claim 15, wherein the plasticizing agent includes a polyalkylene glycol.

20. The method of claim 15, wherein the plasticizing agent includes a quaternary ammonium salt.

21. The method of claim 15, wherein the applying includes applying the dermal covering with a SAMMS sorbent dispersed in a gel-forming polymer.

22. The method of claim 15, wherein the applying includes applying the dermal covering with a member selected from the group consisting of: a bandage, a gauze, a sponge, and combinations thereof.

23. The method of claim 15, wherein the applying includes applying the dermal covering with an adhesive agent that adheres the dermal covering to the dermal surface.

24. The method of claim 15, wherein the applying includes applying the dermal covering with an absorbing agent that absorbs exudates from the dermal surface.

25. The method of claim 15, wherein the applying includes applying the dermal covering with a member selected from the group consisting of: a wound healing agent; an antimicrobial agent; a hemostatic agent, a steroid, an anti-inflammatory agent, and combinations thereof.

26. The method of claim 15, wherein the plasticizing agent allows detachment of the dermal covering from the dermal surface at a lower pain or irritation threshold compared to detachment without the plasticizing agent.

27. The method of claim 15, further including the step of detaching the dermal covering containing radionuclides from the dermal surface, reducing the radiological burden on the dermal surface.

28. The method of claim 27, wherein the method is performed one or more times.
